(19) United States
(12) Patent Application Publication
(10) Pub. No.: US 2010/0280419 A1
(43) Pub. Date: Nov. 4, 2010

(54) Ultrasonically Assisted Fitness Method and Apparatus

(76) Inventors: Dimitri Donskoy, Fair Haven, NJ (US); Tatyana Donskoy, Fair Haven, NJ (US); Nikolay Sedunov, Jersey City, NJ (US)

Correspondence Address: GREENBERG TRAURIG, LLP, 200 PARK AVE., P.O. BOX 677, FLORHAM PARK, NJ, 07932, US

(21) Appl. No.: 12/768,986

(22) Filed: Apr. 28, 2010

Related U.S. Application Data
(60) Provisional application No. 61/174,604, filed on May 1, 2009.

(51) Int. Cl. A61N 7/00

(52) U.S. Cl. 601/2

(57) ABSTRACT

A device for accelerating the loss of regional body fat tissue in an exercising person, which includes a plurality of ultrasonic transducers for emitting ultrasound, and an implement for containing the transducers which is positioned proximate to the regional body fat tissue in the exercising person. The device includes means for controlling the transducers, whereby said ultrasound enhances the loss of the regional body fat tissue in the exercising person when the device is positioned proximate the regional body fat tissue and ultrasound is admitted thereto.

### CROSS-REFERENCE TO RELATED APPLICATIONS

The present application claims the benefit of U.S. Provisional Patent Application Ser. No. 61/174,604, filed May 1, 2009, which is incorporated by reference herein in its entirety.

### FIELD OF THE INVENTION

The present invention relates to a physical fitness device and, more particularly, devices for reducing fat deposits in particular areas of the body through the combined use of directed ultrasonic energy and physical activity.

### BACKGROUND OF THE INVENTION

Almost 130 million adults in the U.S. are overweight, over 60 million are obese and about 10 million severely obese. Obesity is the second leading cause of preventable death in the U.S. and is on the verge of surpassing the first cause. The number of body-conscious people looking for a silver bullet in solving the problem is growing. A number of unhealthy as well as phony solutions (such as electrical stimulation of muscles, etc.) have been offered in the weight-loss market claiming that one can lose weight while resting or even sleeping—such claims have been pronounced fake by respected relevant organizations including the FTC (Federal Trade Commission). In numerous reports, including reports by the FDA (Food and Drug Administration), physical activity is a key part of maintaining weight loss. However, exercise requires significant will and effort which overweight people often lack. The reason is that due to slow metabolism in such individuals, the substantial effort brings very little immediate visible results and these people are willing to give up too soon.

In the course of an exercise, the excess body fat is removed more rapidly from some parts of the body than from others. For example, contrary to the desire, most of the weight loss may come from the upper part of the body such as chest before any reduction is observed in middle-low parts such as thighs, hips, low abdomen, or waist. This result is undesirable aesthetically and psychologically; the body does not attain proper proportions. Therefore, it is essential to offer an assisting tool to support the people's healthy exercise habit which could make their effort more effective and efficient and produce the differential reduction in body fat deposits, while encouraging them to continue the healthy habit of physical activity and exercise.

There are numerous ultrasonic as well as low frequency vibration massaging devices which are marketed as fat reducing devices. Although there is no solid scientific evidence, these devices may produce a marginal effect. There is also ultrasonic liposuction which is a medical procedure utilizing high intensity ultrasonic energy which destroys fat cells through destructive ultrasonic cavitation processes. However, no devices call for the fundamental thermodynamic principle of energy conservation. That is, fat cells are a stored energy source: if they are not removed mechanically through liposuction or distraction, they can only be reduced in size by releasing the stored energy through bio-chemical reactions naturally stimulated by the increased energy demand. Such a demand normally happens during starvation or physical activity, which are the key factors in fat reduction. The released energy must be consumed and it is consumed through physical activity, otherwise the excess energy is recycled back to storage (fat). The claims that a person with a normal diet sitting in a chair, applying various treatments such as electrical muscle stimulation, crème, ultrasound or vibration and effectively loosing weight is merely a marketing claim and has been rejected by numerous governmental actions. Some of these measures could indeed be quite helpful only if combined with sufficient physical activity.

### SUMMARY OF THE INVENTION

The present invention overcomes the disadvantages and shortcomings of the prior art discussed above by providing a new and improved method and apparatus for achieving a selective regional loss in body fat tissue during and/or shortly after a fitness exercise or equivalent physical activity. The reduction of those fatty deposits, occurring in the targeted areas which normally reduce more slowly, may be accelerated. With the use of the methods and apparatus disclosed herein, a person undergoing a weight control program may effectively and efficiently achieve desirable body proportions.

In accordance with the present invention, ultrasonic vibrations are selectively delivered to a portion of the body where fat tissue reduction is desired. The ultrasonic vibrations operate on molecular and blood capillary levels significantly accelerating fat catabolism in fat tissues during and/or shortly after a fitness exercise session. In this manner the reduction of fat tissue, that may be difficult or impossible to obtain otherwise, is accelerated and a balanced and proportional body shape is achieved.

More particularly, unlike low frequency vibrations, applied ultrasonic waves operate on molecular and blood capillary levels stimulating body sympathetic nerves and cells; causing movement of cytoplasm, rotation of mitochondria, and vibration of the cell nucleus. It stimulates and expands the cell membrane and improves local blood and lymph circulation. Thus, the direct stimulation of the sympathetic nerves leads to production of a hormone activating hormone-sensitive lipase (the enzyme that breaks down fat into fatty acids and glycerol to be used as energy thought the body). Ultrasonically enhanced capillary blood and lymph circulation increases the delivery of the hormones to the targeted parts of the fat storage triggering the fat break-down processes, further enhanced by ultrasonic stimulation of cells and circulatory mechanisms. All of these significantly accelerate fat catabolism in targeted fat tissues during a general or targeted fitness exercise encouraging reduction of fatty tissue in selected portions of the body having a regional body fat deposit, so that a selective acceleration in reduction of the regional body fat deposit is achieved.

### BRIEF DESCRIPTION OF THE DRAWINGS

For a more complete understanding of the present invention, reference is made to the following detailed description of exemplary embodiments considered in conjunction with the accompanying drawings, in which:

FIG. 1A is a rear perspective view of a person wearing a garment with ultrasonic transducers built-in;

FIG. 1B is a front perspective view of the person shown in FIG. 1A wearing the garment with ultrasonic transducers built-in;

FIG. 2 is a rear perspective view of one of the ultrasonic transducer module shown in FIGS. 1A and 1B with magnetic connectors exposed;

FIG. 3 is a schematic drawing of the transducer module shown in FIG. 2;

FIG. 4 is a front perspective view of a person wearing waist and thigh belts with ultrasonic transducers built-in;

FIG. 5 is a side elevational view of a thigh exercise machine with ultrasonic transducers attached thereto;

FIG. 6 is a front perspective view of an abdominal exercise machine with ultrasonic transducers attached thereto;

FIG. 7 is a perspective view of an ultrasonic belt with ultrasonic transducers magnetically connected thereto, one ultrasonic transducer being shown detached to expose magnetic connectors;

FIG. 8A is a top plan view of an ultrasonic transducer configured to increase area coverage; and

FIG. 8B is a side elevational view of the ultrasonic transducer shown in FIG. 8A.

### DETAILED DESCRIPTION OF THE EXEMPLARY EMBODIMENTS

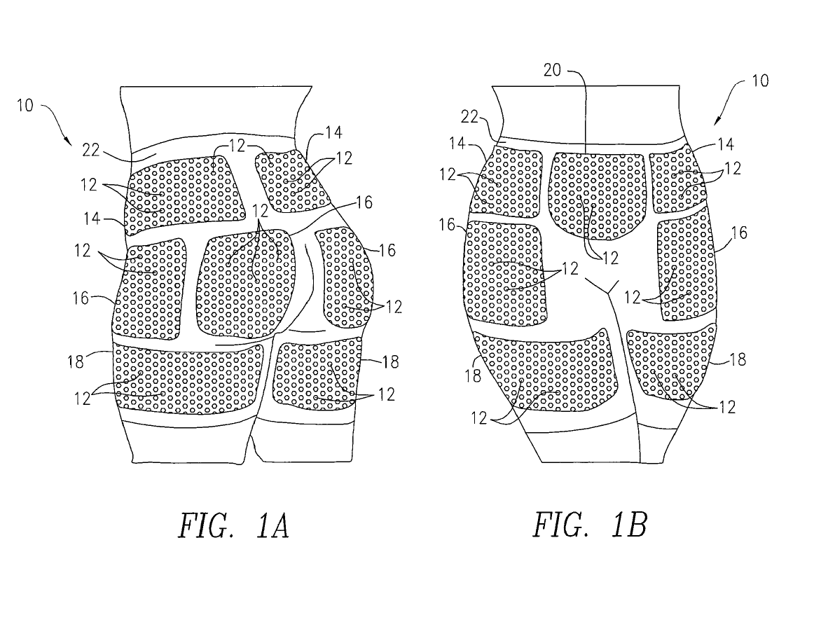

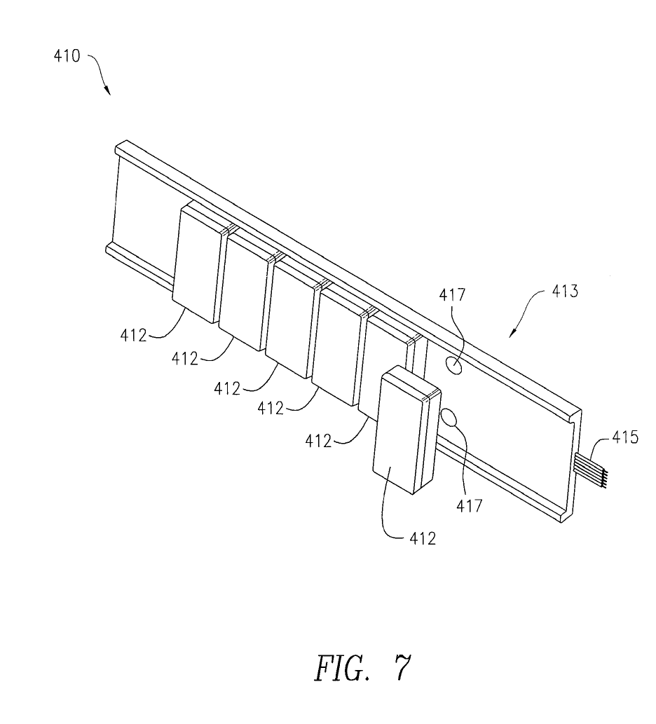

The present invention provides apparatus and methods that apply ultrasonic vibrations to a person's regional body fat tissue during the time the person is exercising. FIGS. 1A and 1B illustrate an ultrasonic wave fitness and fat reduction device (i.e., “the device 10 ”) constructed in accordance with an exemplary embodiment of the present invention. The device 10 has ultrasonic transducers 12 arranged in arrays 14-20 (i.e., 14, 16, 18, and 20). The ultrasonic transducers 12 are arranged in symmetrical or asymmetrical patterns on a flexible material such as a mesh (not shown) which also arranges wiring (see 415 in FIG. 7) for the ultrasonic transducers 12. In an embodiment, the arrays 14-20 are built-in or permanently attached to an implement 22 such as a garment (i.e., as shown in FIGS. 1A and 1B) or may be removable from the implement 22. In an embodiment, the ultrasonic transducers 12 may be attached directly to the implement 22.

When the device 10 is worn by the user during and shortly after an exercise, and depending on a desired outcome, all or selected transducer arrays 14-20 targeting only desired areas may be energized or turned on. For example, if a person desires to reduce only waistline fat tissue, only the array 14 is turned on. Each one of the arrays 14-20 are sized and arranged to target a specific area of the body with excess fat tissue. More particularly, the array 14 targets the waist, the array 16 targets the hips and buttocks, the array 18 targets the thighs, and the array 20 targets the lower abdomen.

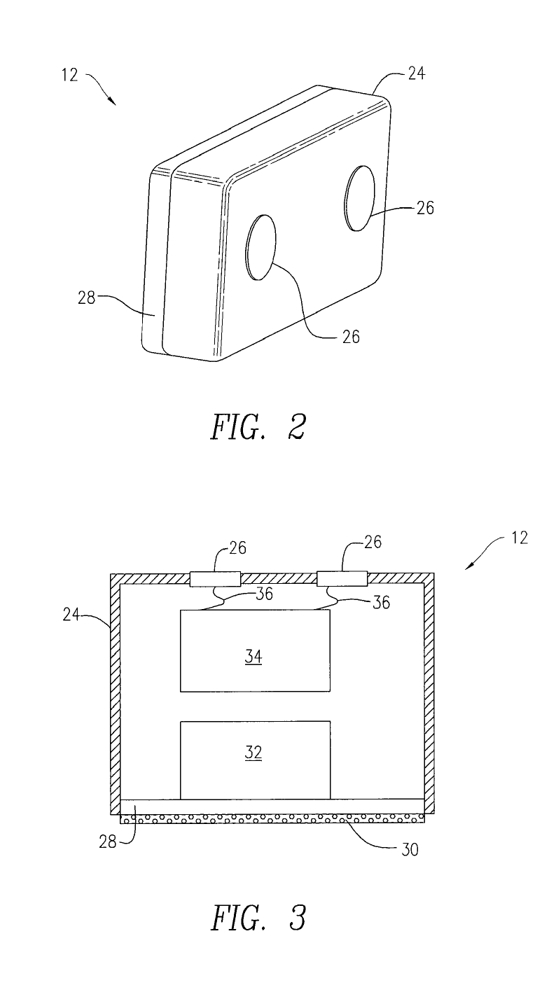

Referring now to FIGS. 2 and 3, each ultrasonic transducer 12 comprises a housing 24 with built-in mechanical and electrical connecting means 26 such as magnets (i.e., as shown in FIGS. 2 and 3), and a skin contact surface 28 for contacting the skin of the user (not shown) to transmit ultrasonic waves through the skin toward the fat tissues. The contact surface 28 contacts the skin through a coupling agent such as a gel, an ointment or a cream (not shown) preferably via a removable pad 30 (see FIG. 3) which may be reusable or disposable. More particularly, the coupling agent can be implemented using the pad 30 having a spongy layer (not shown) saturated with the coupling agent and placed between the skin contact surface 28 and the skin. The pad 30 may have an adhesive coating (not shown) for convenient attachment to the ultrasonic transducers 12 and/or the skin of the user. An inflatable garment or belt (not shown) may be incorporated in the pad 30 to enhance adhesion between the ultrasonic transducers 12 and the skin.

Referring specifically to FIG. 3, the ultrasonic transducer 12 has an ultrasonic actuator 32 which is controlled by electronic circuitry 34 which is powered by a power supply (not shown) connected to the connecting means 26 by wiring 36. The electronic circuitry 34 communicates with a user interface (not shown) and activates the ultrasonic actuator 32 based on signals received from the user interface.

The ultrasonic actuator 32 vibrates at an ultrasonic wave frequency or multiple frequencies in response to an output signal from the electronic circuitry 34, transmitting ultrasonic waves through the skin contact surface 28 via the coupling agent on the removable pad 30 to the body of the user. The circuitry 34 controls the wave form (continuous or burst) of the ultrasonic vibrations, its duration, the repetition frequency if used, pulse/burst vibration, vibration intensity and frequency. These parameters could be pre-programmed. It could be one or a number of pre-set programs each of which is designed for a specific exercise or activity. All or some of these parameters or programs can be manually adjusted or switched by a person using the user interface. The interface could be of various complexities: from simple buttons to more sophisticated LCD display with soft buttons, or voice activated interface. The interface and the electronic circuitry 34 may control one or multiple transducers 12 depending on the configuration of the arrays 14-20.

The ultrasonic transducers 12 may utilize frequencies from about 20 kHz to about 5 MHz. The preferred frequency range is 0.5 MHz to 3 MHz. The electronic circuitry 34 may have a switch between lower and higher frequency: the lower frequency is chosen for thicker fat layers; the higher frequency is for thinner fat layers. For example, 3 MHz frequency is used for a fat layer not exceeding 2 cm in thickness, while 1 MHz frequency is used for the layers of 2 to 5 cms in thickness.

The ultrasonic transducers 12 may be energized using portable energy sources such as batteries or line electric power. The ultrasonic transducers 12 could be also energized using power harvested from the exercising apparatus (not shown) by using electro-mechanical energy harvesting devices (not shown).

The ultrasonic coupling between the ultrasonic transducers 12 and the body facilitated by the contact agent (i.e., a gel, an ointment, or a crème placed on the skin contact surface 28 of the ultrasonic transducer 12) may be combined with a substance with active ingredients specifically designed for fat tissue reduction, such as aminophylline or similar substances, to further enhance the desired effect of fat tissue reduction. In this regard, the effects of ultrasonic stimulation of fat cell reduction will be combined and amplified by the active ingredients. The ultrasound, in turn, enhances the transdermal delivery of such active ingredients due to the effect of sonophoresis. Therefore, the combination of ultrasonic vibrations and the coupling substance with active fat reducing ingredients will significantly enhance the overall desired effect of fat tissue reduction.

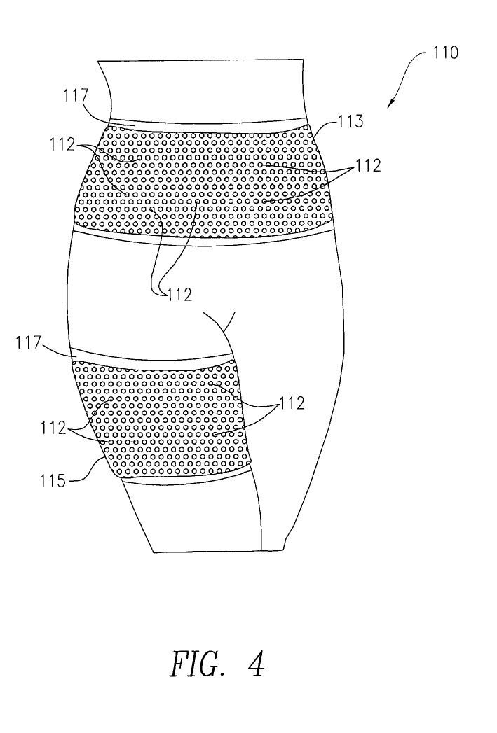

FIGS. 4 through 8B depict second, third, fourth, fifth, and sixth embodiments, respectively, of the present invention. Elements illustrated in FIGS. 4 through 8B, which correspond, either identically or substantially, to the elements described above with respect to the embodiment shown in FIGS. 1A through 3 have been designated by corresponding reference numerals increased by one hundred, two hundred, three hundred, and four hundred, respectively. Unless otherwise stated, the embodiments shown in FIGS. 4 through 8B are constructed and assembled in the same basic manner as the embodiment shown in FIGS. 1A through 3.

Referring to FIG. 4, a device 110 is constructed in accordance with a second exemplary embodiment of the present invention. The device 110 comprises ultrasonic transducers 112 arranged in arrays 113, 115. The arrays 113, 115 may be attached, singly or together to an implement 117 such as a belt or strip (i.e., as shown in FIGS. 1A and 1B), or may be removable from the implement 117. The arrays 113, 115 may be attached directly to the body of the user. The array 113 targets the waist and lower abdomen, and the array 115 targets the thighs.

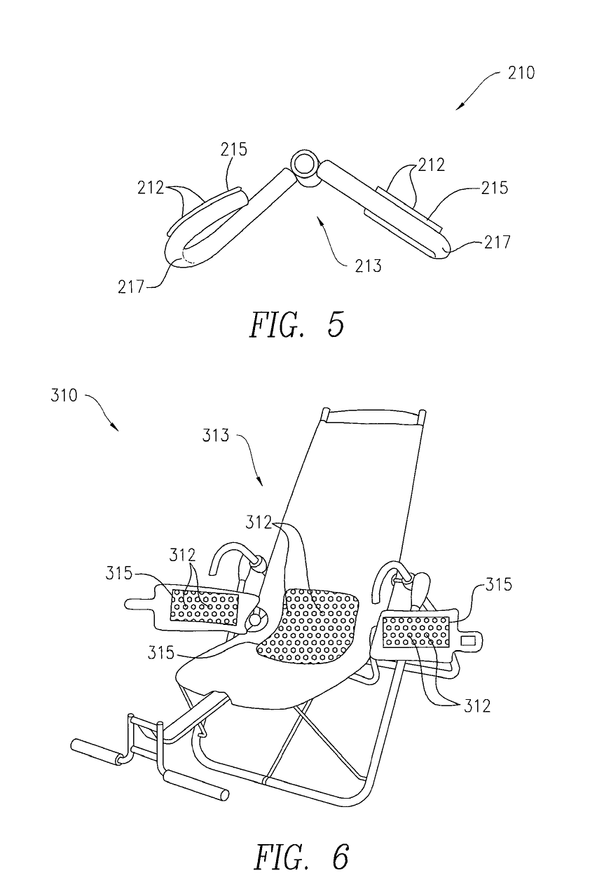

FIG. 5 shows a device 210 constructed in accordance with a third exemplary embodiment of the present invention. The device 210 comprises an implement 213 (e.g., a thigh exercise machine) with ultrasonic transducers 212 arranged in arrays 215 attached thereto. More particularly, the implement 213 has arms 217 on which the arrays 215 are attached (e.g., using Velcro™ or other means not shown) and positioned to directly contact with the body of the user (i.e., press against the body). The pressure of the body contact, in conjunction a contact agent such as gel, ointment or crème (including the ones with active ingredients designed to cause the fat cells to shrink), provides good body coupling for ultrasonic energy.

FIG. 6 shows a device 310 constructed in accordance with a fourth exemplary embodiment of the present invention. The device 310 includes an implement 313 (e.g., an abdominal exercise machine) with ultrasonic transducers 312 arranged in arrays 315 attached thereto. The arrays 315 may be pressed against the body of the user while he or she is sitting in the implement 313 during and after exercise.

FIG. 7 shows a device 410 constructed in accordance with a fifth exemplary embodiment of the present invention. More particularly, the device 410 has a modular design which includes an plurality of ultrasonic transducers 412 removably attached to an implement 413 such as an elongated band or belt. The implement 413 has built-in wiring 415 connected to connecting means 417. More particularly, the connecting means 417 may be magnets built into the implement 413 to facilitate the mechanical attachment of the ultrasonic transducers 412 to or from the implement 413. The connecting means 417 also facilitates electrical connection for powering and communicating with the ultrasonic transducers 412. The implement 413 is connected to a battery or line power supply (not shown) and a user interface unit (not shown) for turning the belt with ultrasonic transducers 412 on and off, as well as setting up a program for the controlling the operation of the ultrasonic transducers 412 (i.e., setting parameters such as output intensity, duty cycle, duration of the sonification, etc.).

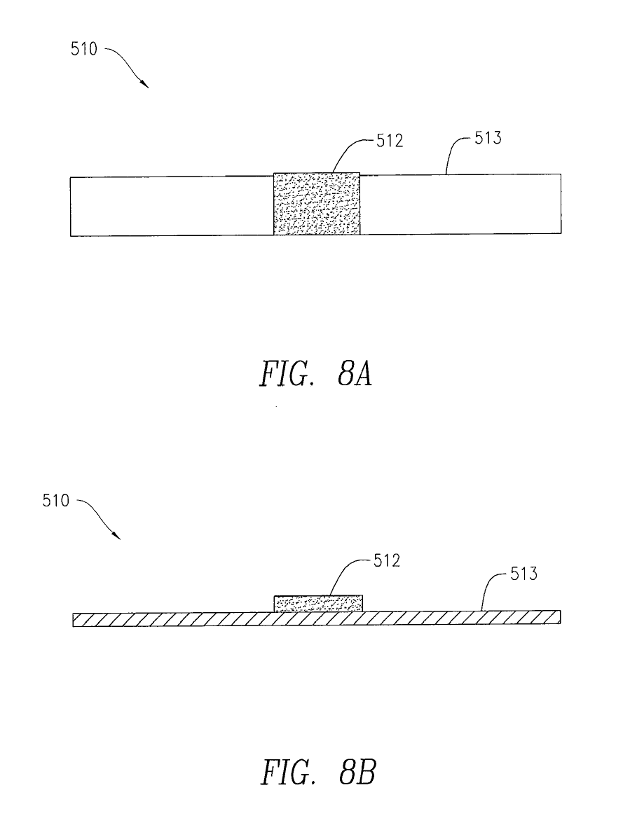

FIGS. 8A-8B shows a device 510 constructed in accordance with a sixth embodiment of the present invention. The device 510 has a skin contact surface 513 which is sized and shaped in the form of a band-like ultrasonic waveguide/resonator for enhanced coverage area for sonification. An ultrasonic transducer 512 is attached to the skin contact surface 513. The skin contact surface 513 can be made of flexible metal, plastic or composite plate which can be tuned to resonate for at specific ultrasonic frequencies for enhanced insonification. The tuning of the skin contact surface 513 depends on its geometry (e.g., thickness and length) and its material properties (e.g., density and elastic moduli). A number of devices 510 can be arranged into an array (not shown) for greater coverage area for sonification.

In operation, the ultrasound intensity should not exceed the therapeutic ultrasound safety limits established by the respective government authorities, such as the FDA. Currently, this limit should not exceed 1 W/cm² at 1 MHz frequency. The preferred ultrasonic intensity in the preferred frequency range should be on the range of 0.01-1 W/cm². The intensities below 0.01 W/cm² also could be used with limited effectiveness.

It is known that prolonged exposure to ultrasound energy of sufficient intensity could induce tissue temperature rise which may cause undesirable and damaging effects. The temperature rise is dependent on several factors, including tissue properties (e.g., absorption coefficient, density, perfusion, etc.), ultrasound exposure parameters [e.g., frequency, pressure amplitude, pulse duration, pulse repetition frequency (PRF), etc.], and beam and array configurations. Thus, the generation of heat in tissues must be controlled through proper exposure design.

For non-focused exposure, the maximum temperature rise occurs in the thin layer of skin in direct contact with an ultrasonic transducer. The temperature rise due to ultrasonic heating not exceeding 1.5° C. is considered safe for practically unlimited exposure time. For 2° C. temperature rise the exposure time should be limited to 60 min, and for 3° C. temperature rise the exposure time should be limited to 15 min. The studies (refer to M. G. Curley, “Soft Tissue Temperature Rise Caused by Scanned, Diagnostic Ultrasound” IEEE transactions on ultrasonics, ferroelectrics, and frequency control, Vol. 40, No. 1, 1993 ) showed that 1° C. temperature rise for a liner transducer array transmitting continuously is determined by the formula (210 mW*MHz/cm)*L/f, where f is the ultrasonic frequency in MHz, and L is the length of the linear transducer array in cm. This formula in combination with safe exposure duration guidance cited above can be used to evaluate the exposure duration for continuously applied acoustic intensity at given frequency:

| Frequency, | Acoustic Intensity, | Max. |
|---|---|---|
| MHz | mW/cm² | exposure time |
| 1 | 300 | unlimited |
| 1 | 400 | 60 min |
| 1 | 600 | 15 min |
| 2 | 600 | unlimited |
| 2 | 800 | 60 min |
| 2 | 1000 | 15 min |
| 3 | 900 | unlimited |
| 3 | 1200 | 60 min |
| 3 | 1800 | 15 min |

In the aforesaid exemplary embodiments of the present invention, various ultrasonic waveforms can be employed: continuous waveform with fixed or varying frequency, multi-frequency, or the pulse ultrasound with fixed or changeable repetition rate. For pulse ultrasound exposure, the above exposure time must be adjusted respectively.

The present invention utilizes the fitness level ultrasonic energy which is much below the cavitations threshold within the limits of therapeutically acceptable intensities defined by the medical community and respective government authorities, such as the FDA. At this level, the ultrasound does not destroy cells, it accelerates fat catabolism. However, the use of fitness ultrasound alone may not produce the appreciable and desirable fat reduction effect. It will do so in combination with physical activities: be it general physical exercise, targeted exercise, or routine physical activity of equivalent intensity/duration, such as raking leaves, shoveling snow, etc. The application of the fitness ultrasound must be during physical activity requiring extra energy consumption taken from the fat deposits.

The application of the fitness ultrasound can be also done shortly after intense physical activity. This is because the metabolism remains elevated for several hours for very heavy exercise (i.e., per to Jack H. Wilmore, Exercise, Obesity, and Weight Control; Series 1, Number 6, of The President's Council on Physical Fitness and Sports (PCPFS) Research Digest).

The desirable fat tissue reduction could be further enhanced by topical application to the skin as in an ointment or crème containing active ingredients designed to cause the fat cells to shrink by causing them to release lipid fat particles. It was documented that certain active ingredients such as aminophylline may accelerate fat tissue reduction. However, transdermal delivery of such active ingredients is limited due to poor penetration trough skin. On the other hand, ultrasonic waves of certain frequencies and intensities significantly improve transdermal delivery of drugs and skin rejuvenating substances. Such a phenomenon is often referred as sonophoresis.

The combined application of fat reducing crème or gel and ultrasonic vibrations will serve two purposes: crème will serve as a contact agent for better coupling of the ultrasonic vibration to the skin; ultrasound will significantly enhance transdermal penetration of the crème's active ingredients, ultrasonic vibrations will increase capillary blood supply to targeted area. Together, ultrasound and active ingredients will amplify the fat reducing effect from each other significantly improving overall effectiveness of approach.

With a few exceptions, physical exercise is carried on spontaneously even in fitness centers. In cases when a client works out with an individual trainer, the latter develops an individual program which makes it organized and personalized for one particular series of sessions, but even in such cases it's done through verbal/visual monitoring; therefore there is no record of quantitative measurement and the client has no history of what is effective for him/her and what's not. The proposed program is designed to organize relevant individual physical measurements and the history of physical activity into Fitness History Chart similar to a medial record.

A fitness center, sport club, or individual training client's information is entered into the program: information such as name, gender, age, previous fitness history, previous diet history and other relative info (may be altered by the user, i.e. fitness instructor and the like). The most important part is quantitative information about the client: weight history, BMI, fat layer(s) measurements in specific areas, C-W-H (chest-waistline-hips) ratio history. Most likely, a new client would not have a measurement history (probably except for the weight). Then the client will be measured similarly to the measurement at the first visit to a doctor's office, and then re-measured at least once a year at his/her suggested annual “check-ups”. The client will be given a recommendation from the instructor—this fitness regimen is to be entered into the “chart” similarly to the paperwork procedure at a doctor's office. The information will be kept in an individual client's file in the program similar to a medical record therefore creating a Fitness Record. The program will allow monitoring the progress of the fitness activity through reports, graphs, and diagrams; it will be possible to observe the progress within any chosen period of time, or any measurable data. It will also be used for modification of the fitness program to achieve the desirable outcome faster and more efficiently for a particular individual. For example, the client may choose to inquire the history of fat loss in the abdominal area for the last 3 months within his/her exercise regimen recommended by the instructor. The information will appear in the form of a report (table), a graph and a diagram. The instructor may modify the frequency, load factors, sequence and type of excises to accelerate the desirable outcome.

It should be appreciated that the present invention provides numerous advantages over the prior art discussed above. For instance, the device 10 encourages the reduction of fatty tissue in selected portions of the body having a regional body fat deposit. The device 10 enables enhanced, convenient, and selective reduction of regional body fat deposits in targeted fat tissues during a general or targeted fitness exercise.

It should be noted that the present invention can have numerous modifications and variations. For instance, increased coverage area for the application to the device 10 may be accomplished by utilizing electromagnetic transducers comprising of an elastic plate, electric coil (or patterned electrode) energized at the excitation frequency and source of constant magnetic field.

The device 10 may also be adapted to provide instantaneous feedback to the user, during an ultrasonically assisted fitness program. This can be achieved by using ultrasonic signals for direct measurements of the thickness of the fat and muscle layers. More particularly, modified ultrasonic transducers may be used for measurements of the thickness by switching them into a measuring mode. The measurements may be accomplished using time-of-flight measurements of a short ultrasonic burst. For example, using 0.2 μs burst with 5 MHz central frequency, and considering the speed of sound in fat tissue to be 1540 m/s, the fat layer thickness could be measured with 0.3 mm accuracy.

It will be understood that the embodiments described herein are merely exemplary and that a person skilled in the art may make many variations and modifications without departing from the spirit and scope of the invention. All such variations and modifications are intended to be included within the scope of the invention as defined in the appended claims.

We claim:

1. A device for accelerating loss of regional body fat tissue in an exercising person, comprising:
at least one transducer for emitting ultrasound;
an implement containing said at least one transducer and which is positioned proximate to the regional body fat tissue in the exercising person; and
controlling means for controlling said at least one transducer, whereby the ultrasound emitted by said at least one transducer accelerates the loss of the regional body fat tissue in the exercising person when said implement is positioned proximate to the regional body fat tissue and ultrasound is admitted thereto.

2. The device of claim 1, wherein said implement includes a contact substance.

3. The device of claim 1, wherein said implement includes a belt.

4. The device of claim 1, wherein said implement includes a garment.

5. The device of claim 1, wherein said implement is imbedded into exercise equipment.

6. The device of claim 1, wherein said implement includes a fat tissue thickness measurement device that provides immediate measurement results.

7. A method for accelerating loss of regional body fat tissue in an exercising person, comprising the steps of:
selecting an exercise routine;
securing an implement containing at least one transducer for emitting ultrasound and in which said implement is positioned proximate to the regional body fat tissue in the exercising person; and
providing controlling means for controlling said at least one transducer, whereby said ultrasound accelerates the loss of the regional body fat tissue in the exercising person when said implement is positioned proximate to the regional body fat tissue and ultrasound is admitted thereto.
